(19) United States
(12) Patent Application Publication
(10) Pub. No.: US 2022/0115142 A1
(43) Pub. Date: Apr. 14, 2022

(54) SYSTEM AND METHOD FOR TRACKING A USERS HEALTH STATUS

(71) Applicant: Healthcare Integrated Technologies Inc., Knoxville, TN (US)

(72) Inventors: Kenneth M. GREENWOOD, Davenport, FL (US); Scott Michael BORUFF, Knoxville, TN (US); Jurgen VOLLRATH, Sherwood, OR (US)

(21) Appl. No.: 17/500,885

(22) Filed: Oct. 13, 2021

Related U.S. Application Data
(60) Provisional application No. 63/204,620, filed on Oct. 14, 2020.

(51) Int. Cl. G16H 50/30; G16H 50/20; G16H 10/20; G16H 10/60; G16H 80/00

(52) U.S. Cl. CPC G16H 50/30; G16H 50/20; G16H 80/00; G16H 10/60; G16H 10/20

(57) ABSTRACT

In a system for monitoring a user's health and intervening in the event of a health issue being identified, patient information is captured through a combination of user self-assessment and health-sensor data, thereby allowing early diagnosis and improving the health of patients and limiting the spread of infectious diseases.

### FIELD OF THE INVENTION

The invention relates to medical care. In particular it relates to preventative medicine, identifying medical problems early, and avoiding readmissions.

### BACKGROUND OF THE INVENTION

Much of the cost associated with medical care is that it focuses on fixing problems once they have occurred. Often a patient will only see a physician once their condition has become acute, thereby increasing the difficulty and cost associated with curing the patient.

Another aspect that drives up medical costs is readmission of a patient following a procedure.

The present invention therefore seeks to ensure early identification and intervention to ensure the improved health of patients, as well as compliance management in order to reduce readmissions, all directed to reducing medical costs.

### SUMMARY OF THE INVENTION

The present invention applies to both healthy patients, and sick or recently treated patients. In the case of healthy patients, it serves to implement a preventative and early detection approach to ensure the wellbeing of a patient. In the case of sick or recently treated patients, it ensures compliance with a care plan (also referred to herein as a care pathway).

According to one aspect of the invention, a patient's health information is monitored and analyzed for potential health issues.

Thus, according to the invention, there is provided a method for monitoring a patient's health, which comprises, capturing the patient's health information on an ongoing basis, whether or not a prior health issue has been identified, identifying health concerns, and generating a response in the event of an identified health concern.

The invention may also comprise a method for monitoring a patient's health, that includes capturing the patient's health information on an ongoing basis using online patient self-assessments and patient sensor data that are both uploaded to a processor, identifying health concerns, and generating a notification to one or more persons in the event of an identified health concern.

The capturing of a patient's health information on an ongoing basis, may include daily or continuous health information captures.

Health information may include one or more of user self-assessments by the patient, and sensor data.

Self-assessments may be template driven, e.g. a patient may be presented with a user interface on a computing device such as a tablet, smart phone or laptop.

The patient's self-assessments may be supplemented with the sensor data, which may include the use of permanent monitoring devices of patient, and/or ad hoc medical sensors applied to or used on a patient for the duration of the assessment.

Permanent monitoring devices may include wall-mounted image capture devices such as video cameras, lidar, radar, etc. and wearables such as pendants, wrist bands and clothing that includes sensors.

Identifying health concerns may comprise one or more of: comparing health information received from a patient with previously stored third-party data about abnormal conditions in order to identify similarities to abnormal conditions, and monitoring health information received from a patient to identify anomalies over time. The information received from the patient as part of self-assessment may be supplemented with electronic medical record (EMR) information about the patient to identify changes in the patient's health since the last official medical appointment.

A notification generated in the event of an identified health concern may comprise notifying the patient, e.g., that they should see a physician about an identified concern; notifying medical practitioners or support staff associated with the patient, of a potential health concern; notifying family members, next of kin or emergency contact persons associated with the patient, or notifying authorized persons or authorities of potential health concerns that may impact other people.

Further, according to the invention, there is provided a method of monitoring a patient's health, comprising monitoring at least one of: the patient's physiological parameters, and compliance by the patient with a care plan, wherein monitoring the patient's physiological parameters includes capturing the patient's health information on an ongoing basis using at least one of: online patient self-assessments and patient sensor data, and wherein monitoring for compliance by the patient with a care plan includes at least one of: online patient self-assessments and sensor-based compliance data, wherein all patient self-assessment data and all sensor data is uploaded to a processor, the method further comprising identifying non-compliance with a care plan or patient health concerns, and generating a notification to one or more persons in the event of an identified non-compliance or health concern.

Further, according to the invention, there is provided a system for monitoring a patient's health, comprising, a patient processing device configured with a patient portal for allowing a patient to enter self-monitoring health information; a processor for analyzing health information received from the patient; a memory configured with machine readable code defining an algorithm for controlling the processor, and communication means for transmitting health information from the patient processing device to the processor. The machine readable code may define an artificial intelligence (AI) system to identifying one or more of: anomalies over time, or similarities to third party data relating symptomology to diagnostics.

The processor and memory may comprise a server, which may be defined by a dedicated server and database or a distributed cloud server such as Amazon Web Services (AWS), or an edge computing system. The processor and memory may instead comprise a local processor and memory, which may be defined by the patient processing device, e.g., by a laptop, tablet or smartphone.

The system may further include one or more sensors or monitoring devices for capturing patient data about the patient. The sensors may also include communication means for transmitting patient data to the processor. The communication means associated with the sensors may comprise Bluetooth or other radio transceivers, e.g. Zigbee, Z-Wave, etc., for communicating with a hub, which may, in turn be connected to the processor via WiFi or other communications channel. The communication means associated with the sensors may also communicate with an edge computing network that defines at least part of the processor function.

The patient processing device may comprise a smart phone, tablet, laptop or desktop computer. The patient portal may be defined by a web app accessed via a browser on the patient processing device, or may be defined by a native mobile application (App) downloaded onto the user processing device.

### BRIEF DESCRIPTION OF THE DRAWINGS

FIG. 1 is a depiction of one embodiment of a system of the invention;

FIG. 2 is an example of one embodiment of a patient self-assessment capture page;

FIG. 3 is a flow chart defining the logic of one embodiment of an anomaly detection algorithm implemented in an AI system;

FIG. 4 is a flow chart defining the logic of one embodiment of an anomaly detection and corroboration algorithm implemented in an AI system, and

FIG. 5 shows one embodiment of the logic involved in contact tracing in accordance with the present invention.

### DETAILED DESCRIPTION OF THE INVENTION

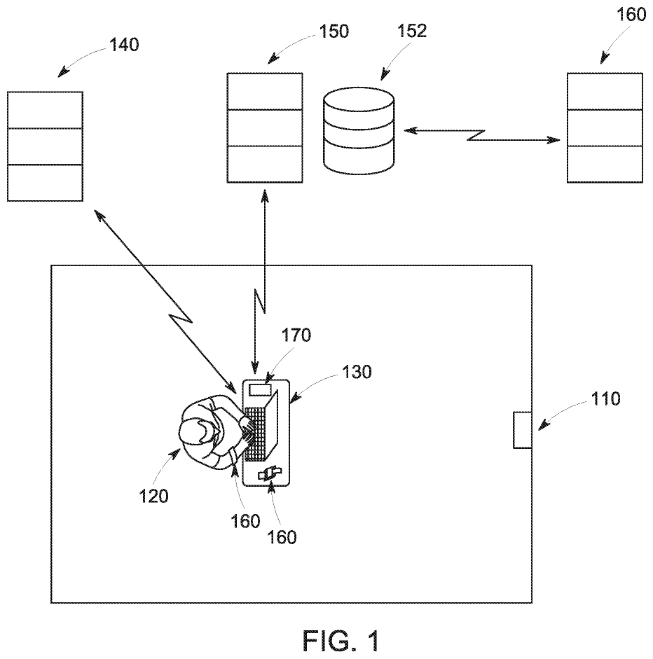

One embodiment of a system of the present invention is shown in FIG. 1.

The patient's apartment 100 in this embodiment, includes a wall-mounted sensor 110.

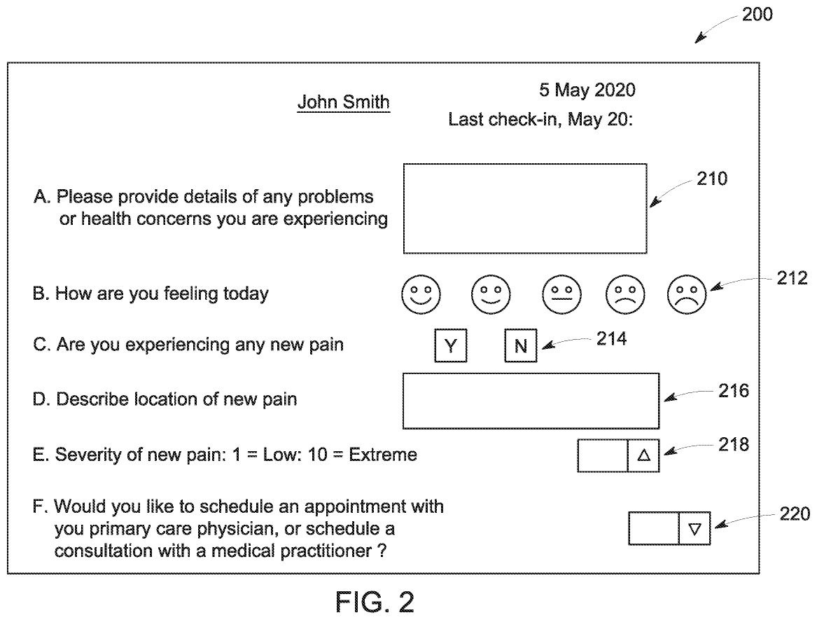

The patient 120 has a patient processing device, which in this embodiment comprises a laptop 130 that has a WiFi connection to the internet via a modem (not shown). The laptop 130 includes a browser allowing the patient to access a web page that defines a patient portal for capturing health information of the patient. The patient portal is discussed in greater detail below with respect to FIG. 2.

The patient portal allows the patient to enter health-related information (also referred to herein as patient self-assessment) on an ongoing basis, e.g., a regular basis, such as once a day or once a week, or the patient may choose to only enter information when they detect a note-worthy event such as a health issue that causes them concern.

The information from the sensor 110 and patient self-assessment is uploaded to a server 140 hosting the web site, or in this case, to another server system 150 with a database 152, for capturing health information and, which includes a memory configured with machine readable code defining an algorithm for monitoring the patient's health. In addition to the self-assessment by the patient using the patient portal, the system can integrate data from other sources, including the patient's electronic medical record (EMR) which may be housed on a physician's or hospital's server 160.

As mentioned above, the patient's apartment 100 includes a wall-mounted sensor 110, which in this embodiment is a radio frequency image capture device or a digital video camera that allows the movements of the patient to be monitored. By capturing this information as part of the health information about the patient, the processor associated with server 150 can monitor the patient's movements and identify anomalies that may indicate a health issue, e.g. failure to move around as much, changes in sleep patterns, excessive times in the bathroom, etc. As discussed above, this anomaly detection is performed by a processor controlled by machine readable code on a memory device connected to the processor. The machine-readable code defines an algorithm which defines the logic performed by the processor. In this case the processor comprises the server 150.

In addition to permanently mounted sensors such as the wall-mounted sensor 110, the patient may also be using other health-related sensors 160, such as a FitBit or other wearables (such as bracelets, pendants or clothing provided with body sensors) or ad hoc health sensors such as blood pressure cuffs or blood oxygenation sensors. These sensors 160 capture different medical or health data about the patient, providing additional information about the patient to supplement the patient's self-assessment information.

The devices 160 may communicate directly with a server or through a hub 170 to a server or to an intermediate, dispersed processor, such as an edge computing system. In this embodiment, the patient's smart phone is in Bluetooth connection with each of the sensors 160 and acts as the communication hub 170 to transmit the patient's health data to a server (in this case server 150) via WiFi or a cell phone connection.

FIG. 2 shows one embodiment of a health information capture page 200 forming part of the patient portal of the invention. In this embodiment the capture page 200 is configured as a web page accessible by the patient via a browser, using a patient processing device such as the laptop 130. It will however be appreciated that the page 200 may instead be downloaded as an App (also referred to as a native local application) onto a smart phone, to define an App based platform, wherein the smart phone acts as the patient processing device.

In this embodiment, the web page 200 comprises a patient information capture page. For ease of description only the health information capture page is shown. It will typically be preceded by a registration page that will capture the basic patient identifying information such as the user's name, email address and other contact information, and typically a user authentication by sending a confirmation message to the patient's specified email address. Once they are registered and log on with their chosen username and password, one of the pages they may access is the health information capture page 200.

The page 200 may request date, time, and location information but typically will auto-generate the date and time information and will have obtained authorization from the patient to access their current location information, which then automatically becomes part of the patient's entry record.

In this embodiment the page 200 is set up as a template-driven page that asks the patient several questions, including:

A. Please provide details of any problems or health concerns you are experiencing (data entry field 210)

B. How are you feeling today? (5 selectable fields 212)

C. Are you experiencing any new pain? (User selection field 214)

D. Describe location of any new pain (data entry field 216)

E. Severity of the new pain: 1=Low; 10=Extreme (data entry with drop-down selection 218)

F. Would you like to make an appointment with your primary care physician or schedule a consultation with a medical practitioner? (provides a drop-down selection to choose one or the other 220)

The patient self-assessment health information page 200 allows the patient to uploaded health information regularly for review or analysis by a processor such as the server 150. By combining this information with sensor information from sensors 110 and 160, the processor can identify anomalies or possible health issues e.g. changes in health conditions of the patient by comparing the defined symptoms and data to a database of medical information associated with health issues (e.g. using data pre-loaded into the database 152).

The processor is controlled by an algorithm on a memory connected to the processor, which compares changes in the patient's data over time, in order to identify anomalies or potential health problems. The changes over time may be based on information obtained not only from the patient during self-assessment sessions, or based on health sensor information that is uploaded to the server 150, but can also take into account information about the patient's last official medical appointment, or the patient's entire health history, by capturing data from the patient's EMR.

In the event of an anomaly being detected that exceeds a defined threshold, one or more authorized or necessary persons are notified, e.g., the patient, the patient's primary physician, next of kin, family members, co-workers, etc., depending on the nature of the medical condition that is diagnosed.

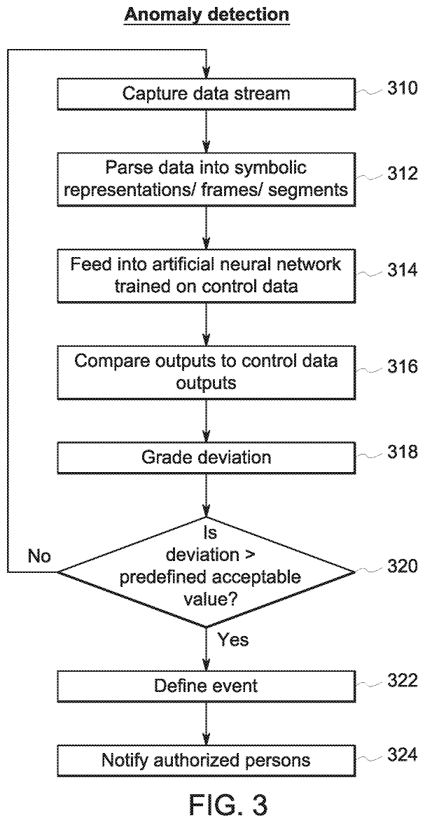

The implementation of such an analysis of the data to identify anomalies over time and similarities to third party data that relates symptomology to diagnostics, requires logic in the form of machine-readable code defining an algorithm or implemented in an artificial intelligence (AI) system, which is stored on a local or remote memory (as discussed above), and which defines the logic used by a processor to perform the analysis and make assessments. One such embodiment of the logic, based on grading the level of the anomaly, is shown in FIG. 3, which defines the analysis based on sensor data that is evaluated by an Artificial Intelligence (AI) system, in this case an artificial neural network. Data from a sensor is captured (step 310) and is parsed into segments (also referred to as symbolic representations or frames) (step 312). The symbolic representations are fed into an artificial neural network (step 314), which has been trained based on control data (e.g. similar previous events involving the same party or parties or similar third-party events). The outputs from the AI are compared to the control data (step 316) and the degree of deviation is graded in step 318 by assigning a grading number to the degree of deviation. In step 320 a determination is made whether the deviation exceeds a predefined threshold, in which case the anomaly is registered as an event (step 322). Similarly, self-assessment data is parsed to extract symptomologies, which are compared to symptomology-diagnostic data to define a patient diagnosis and register the diagnosis as an event if there is a diagnosis corresponding to a health concern. When an even is triggered, one or more authorized persons is notified (step 324).

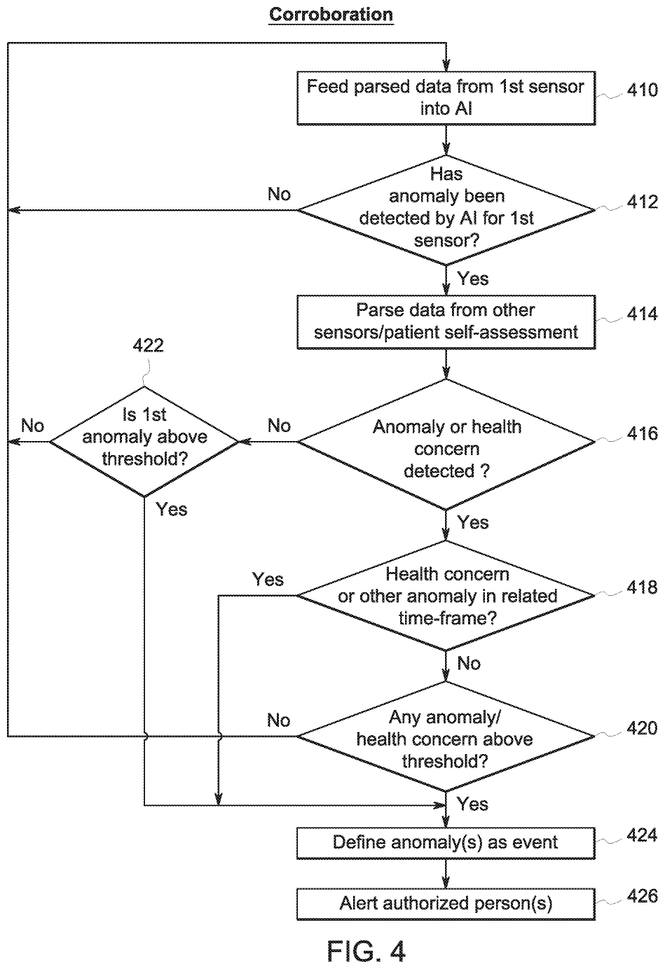

Another embodiment of the logic in making a determination, in this case based on grading of an anomaly and/or corroboration between sensor data or between sensor data and patient self-assessment data, is shown in FIG. 4.

Parsed data from a first sensor is fed into an AI system (step 410). Insofar as an anomaly is detected in the data (step 412), this is corroborated against data from at least one other sensor or patient self-assessment data by parsing data from the other sensors that are involved in the particular implementation or from the patient self-assessment (step 414). In step 416 a decision is made whether any of the other sensor data or patient symptomology from the self-assessment shows up an anomaly or health concern, in which case it is compared on a time scale whether the said anomaly or health concern is in a related time frame (which could be the same time as the first sensor anomaly or be causally linked to activities flowing from the first sensor anomaly) (step 418). If any sensor anomaly or the self-assessment health concern is above a certain threshold (step 420) or, similarly, even if there is no other corroborating data, if the anomaly or health concern from any of the sensors or self-assessment exceeds a threshold value (step 422), a flagging event is triggered (step 424), which alerts one or more authorized persons (step 426).

Another aspect of the invention comprises contact tracing in the event of a patient being diagnosed with an infectious disease—particularly one that is associated with a pandemic or potential pandemic. In the case of an infectious disease that may pose a health risk to others, persons that the patient had contact with (Potential Infectees) at any time during a defined period prior to the diagnosis, can be notified to stay home or self-quarantine and/or get tested. Potential Infectees may, in turn, be monitored for anomalies and subjected to contact tracing to identify persons they may have been in contact with during a defined time prior to the original patient's diagnosis.

Contact tracing may be based on various sources of data, including:

a. Patient identification of persons they were in contact with. This may be captured by the same patient portal that captured the patient health information. Once an infectious disease is identified, the user is presented with a feedback page to describe the current symptoms and requesting that they regularly update this information over the next few days and weeks. They are also asked to list all of the people, location and time and date they were in contact with over the previous 14 days to the best of their abilities.

b. Patient identification is supplemented with location tracking data from patient's cell phone for a defined period, e.g., for 2 weeks prior to diagnosis or prior to the patient being notified and quarantine being secured, e.g., through a patient's smart phone GPS data.

c. Furthermore, the patient's routines and common associations may be captured, including employees, family members, shopping habits, etc., and using this information to identify all of the people that the patient potentially came in contact with for a defined period prior to diagnosis. These patient routines may be based on information provided by the patient through questioning (in person or responding to an on-line questionnaire). The routines may also be based on artificial intelligence analytics of social media activity, emails and other interactions between the patient and third parties, which may be obtained from third party analytics such as those performed by Facebook.

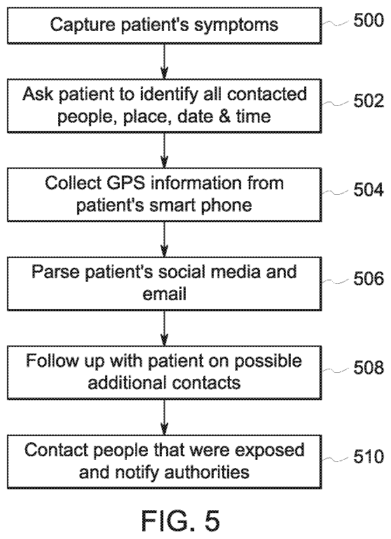

One embodiment of the logic controlling the processor for purposes of contact tracing is shown by way of a flow chart shown in FIG. 5.

In step 500 the patient is asked to describe their current symptoms.

In step 502 they are asked to list all of the people, including location, date, and time they were in contact with over the previous 14 days.

In step 504 the patient's GPS information for the last 14 days is collected.

In step 506 the patient's social media and email information is parsed for people the patient may have been in physical contact with, e.g., information about physical get-togethers.

In step 508 the information from steps 504 and 506 is used to ask the patient about potential contact with said people identified in steps 504 and 506.

In step 510, relevant authorities are notified and people that had contact with the patient are similarly contacted to define their contacts.

While the present application was described with respect to specific embodiments, it will be appreciated that the specific data capture means, layout of capture pages, types of sensors, and methods of analyzing the data, may vary without departing from the scope of the invention.

What is claimed is:

1. A method for monitoring a patient's health, comprising
capturing the patient's health information on an ongoing basis using online patient self-assessments and patient sensor data that are both uploaded to a processor,
identifying health concerns, and
generating a notification to one or more persons in the event of an identified health concern.

2. The method of claim 1, wherein the capturing of a patient's health information on an ongoing basis, includes daily or continuous health information captures.

3. The method of claim 1, wherein the patient self-assessments are template driven, wherein the patient is presented with a user interface on a computing device such as a tablet, smart phone or laptop.

4. The method of claim 1, wherein the sensor data is captured using at least one of: continual monitoring devices associated with the patient or the patient's residence, and ad hoc medical sensors applied to or used on the patient for the duration of the assessment.

5. The method of claim 4, wherein the continual monitoring devices include one or more of: an image capture device mounted in the residence of the patient, and a wearable device.

6. The method of claim 5, wherein identifying health concerns includes one or more of: comparing health information received from the patient with previously stored third-party data about abnormal conditions, and monitoring health information received from the patient to identify anomalies over time.

7. The method of claim 5, wherein the health information received from sensors or the patient self-assessment is supplemented with electronic medical record (EMR) information about the patient to identify changes in the patient's health.

8. The method of claim 1, wherein the notification generated in the event of an identified health concern includes notifying the patient, notifying medical practitioners or support staff associated with the patient, notifying family members or emergency contact persons associated with the patient, or notifying authorized persons or authorities.

9. A method of monitoring a patient's health, comprising
monitoring at least one of: the patient's physiological parameters, and compliance by the patient with a care plan, wherein monitoring the patient's physiological parameters includes capturing the patient's health information on an ongoing basis using at least one of: online patient self-assessments and patient sensor data, and wherein monitoring for compliance by the patient with a care plan includes at least one of: online patient self-assessments and sensor-based compliance data.

10. The method of claim 9, wherein all patient self-assessment data and all sensor data is uploaded to a processor, the method further comprising identifying non-compliance with a care plan or identifying patient health concerns, and generating a notification to one or more persons in the event of an identified non-compliance or health concern.

11. A system for monitoring a patient's health, comprising,
a patient processing device configured with a patient portal for allowing a patient to enter self-monitoring health information;
one or more sensors for capturing data about the patient;
a processor for analyzing health information received from the patient;
a memory configured with machine readable code defining an algorithm for controlling the processor, and
communication means for transmitting health information from the patient processing device to the processor.

12. The system of claim 11, wherein the machine-readable code defines an artificial intelligence (AI) system to identifying one or more of: anomalies over time, or similarities to third party data relating symptomology to diagnostics.

13. The system of claim 11, wherein the processor and memory comprise a server defined by a dedicated server and database, or a distributed cloud server such as Amazon Web Services (AWS), or an edge computing system, or a combination thereof.

14. The system of claim 11, wherein the processor and memory are defined by the patient processing device.

15. The system of claim 11, wherein the sensors include communication means for transmitting patient data to the processor.

16. The system of claim 11, wherein the patient processing device comprises a smart phone, tablet, laptop, or desktop computer.

17. The system of claim 11, wherein the patient portal is defined by a web app accessed via a browser on the patient processing device, or may be defined by a native mobile application (App) downloaded onto the user processing device.
